(19) United States
(12) Patent Application Publication
(10) Pub. No.: US 2019/0350524 A1
(43) Pub. Date: Nov. 21, 2019

(54) Devices, Systems, and Methods for Diagnosis, Prognosis, and Treatment of Anxiety Diseases and Disorders

(71) Applicant: Melissa Hurley, Happy Valley, OR (US)

(72) Inventor: Melissa Hurley, Happy Valley, OR (US)

(21) Appl. No.: 16/399,416

(22) Filed: Apr. 30, 2019

Related U.S. Application Data
(60) Provisional application No. 62/672,135, filed on May 16, 2018.

(51) Int. Cl. A61B 5/00; A61B 5/16; A61B 5/0408

(52) U.S. Cl. CPC A61B 5/6831; A61B 5/165; A61B 5/72; A61B 5/0205; A61B 5/486; A61B 5/6824; A61B 5/6823; A61B 5/0408

(57) ABSTRACT

A device, system, and method for diagnosis, prognosis, and treatment of diseases or disorders, such as psychiatric or psychological disorders characterized at least in part by anxiety. The device includes a heart monitor configured to monitor at least a heart rate of a first individual, and is configured to regulate one or more properties of one or more biofeedback elements according to at least the heart rate of the first individual. The biofeedback elements may include one or more of a light element, a sound element, and a temperature element. The biofeedback elements are incorporated into an initial environment of the first individual to form a resultant environment, which may influence one or more biological processes of the first individual. The system may include a plurality of devices configured to regulate the one or more properties of the one or more biofeedback elements.

### CROSS REFERENCE TO RELATED APPLICATIONS

This application claims priority under 35 U.S.C. § 119(e) to U.S. Provisional Application No. 62/672,135 filed on May 16, 2018. The above identified patent application is incorporated by reference herein in its entirety to provide continuity of disclosure.

### BACKGROUND OF THE INVENTION

The present invention relates to devices, systems, and methods for diagnosis, prognosis, and treatment of diseases and disorders characterized at least in part by anxiety.

Many psychiatric conditions, such as anxiety, can be significant daily obstacles for individuals who suffer from these conditions. Typically characterized by worry, nervousness, or unease, anxiety may manifest as a feeling of helplessness or loss of control over one's self or one's environment. Existing solutions for anxiety include therapy, such as psychotherapy, and medication, which may include one or more tranquilizing agents. Success with therapy is often highly dependent on the nature of the therapist-patient relationship, and if this relationship is lacking, the breadth or depth of the analysis and communication that comprises the therapy may be insufficient to effectively treat the condition. Existing medications designed to treat anxiety may have undesirable side effects, which may include psychological, physical, or emotional changes that may cause suffering to many individuals diagnosed with, or suspected of having, anxiety or a related psychiatric condition.

As a result of these shortcomings, many individuals with anxiety may choose to discontinue therapy or medication. In some scenarios, discontinuation of medication can exacerbate the condition, and resulting crises may include panic attacks or psychiatric events that require hospitalization. The resulting social and economic burdens on society and the healthcare system are significant. There is a need for a more engaging, enjoyable, personal, and effective approach to the treatment of these conditions.

Music therapy is a clinical tool that can be used as part of therapy to treat physical, emotional, cognitive, and social conditions of individuals. Music therapy may involve listening to music as part of a therapeutic regimen, and existing modes of listening to music include selecting a song, collection, or album, and listening to the music as designed and composed by the original artist. The resulting effect may be suitable for certain individuals, however, some individuals suffering from anxiety may experience the music as another element of their environment that is outside their control. This can sometimes add to feelings of helplessness and exacerbate the degree of anxiety experienced.

These feelings, as well as other symptoms of anxiety, often manifest physically as an altered rate of a biological process, such as a heartbeat, which may occur as a result of hyperactive nervous system activity. Individuals having these feelings may be largely unaware of the physical manifestations of their feelings, at least because they are acutely focused on their environment, which may be perceived as a threat or danger. If made aware of the physical manifestations of their feelings, these individuals may be able to gain some degree of control over their feelings to reign in the symptoms and prevent secondary events, such as panic attacks, which may otherwise occur.

Biofeedback includes the conscious monitoring of an unconscious biological process in an attempt to gain control over that process. The systems involved in these procedures are not configured for treatment of individuals with psychiatric conditions, and may be perceived as invasive by patients or found ineffective in many such circumstances by caretakers. Accordingly, biofeedback systems may not be utilized for the diagnosis, prognosis, or treatment of diseases or disorders characterized at least in part by anxiety.

Therefore, there is a need in the art for improved devices, systems, and methods for the diagnosis, prognosis, and treatment of diseases or disorders characterized at least in part by anxiety. The present invention addresses this unmet need.

Devices have been disclosed in the art that relate to biofeedback. These include devices that have been patented and published in patent application publications. These devices are often unsatisfactory or ineffective for treatment of individuals having anxiety. In view of the devices disclosed in the art, it is submitted that there is a need in the art for an improvement to existing biofeedback approaches. In view of the present disclosure, it is submitted that the present invention substantially diverges in structural and functional elements from devices in the art, and substantially fulfills an unmet need in the art.

### SUMMARY OF THE INVENTION

In view of the disadvantages inherent in the known types of biofeedback devices in the art, the present invention provides a new and improved biofeedback device, system, and method, wherein the same can be utilized for providing customized or personalized therapy to an individual suffering from anxiety.

It is therefore an object of the present invention to provide a device, a system comprising the device, and a method of using the device or the system for providing an individual, such as a patient suffering or suspected of suffering from anxiety or a related condition, disease, or disorder, with a tool for effective evaluation, diagnosis, prognosis, and treatment of the anxiety or the related condition. In various embodiments, the device is configured to monitor a biological process of an individual and utilize a rate of the biological process to generate or alter a biofeedback signal that is in turn perceived by the individual.

In various embodiments, the biofeedback signal may include one or more of: light, sound, and temperature. In embodiments having light as biofeedback, one or more properties of the light may be altered as a function of the rate of the biological process, including but not limited to a wavelength or frequency of the light, an intensity of the light, a presence of the light, an absence of the light, or a display pattern of the light. In embodiments having sound as biofeedback, one or more properties of the sound may be altered as a function of the rate of the biological process. For example, if the sound includes music, according to a particular embodiment, a rate of the music corresponds to a rate of the biological process. However, other properties of the music may be altered or controlled alternatively or in addition to the rate of the music, including but not limited to pitch, duration, loudness, timbre, sonic texture, spatial location, song selection, song collection selection, album selection, and the like. In embodiments having temperature as biofeedback, one or more properties of the temperature may be altered as a function of the rate of the biological process, including but not limited to a degree of the temperature (i.e., hot, warm, cool, cold, etc.), an area which is affected by the temperature, a volume which is affected by the temperature, and the like.

Because different individuals suffering from anxiety may each respond differently to the same stimulus, it is envisioned that an individual or a caretaker of the individual may be able to control which stimulus or stimuli are implemented in a particular device, system, method, or design of the present invention. In this manner, a patient who prefers or benefits from light therapy for anxiety may select light as biofeedback, a patient who prefers or benefits from sound or music therapy may select this option, and likewise with temperature therapy. Accordingly, the present invention also provides a highly customizable and personalized diagnosis, prognosis, and/or treatment procedure.

Generally, it is intended that the biofeedback signal(s) (e.g., biosignal(s)) implemented in the present invention be sensed by one or more individuals, for example, at least the individual whose biological process is controlling the biofeedback signal(s). However, in some embodiments, the biofeedback signal(s) may be sensed by additional individuals. Inasmuch as these signal(s) may have the potential or ability to affect one or more biological processes of a second individual who senses them, such sensing by the second individual may be described as a biofeedforward mechanism. In this respect, “biofeedback” may refer to an individual sensing their own biosignal, and “biofeedforward” may refer to an individual sensing a biosignal originating from another individual. In some embodiments, biofeedback of a biosignal is implemented. In other embodiments, both biofeedback of the biosignal and biofeedforward of the biosignal are implemented. In yet other embodiments, biofeedforward may be implemented. Accordingly, based on the present disclosure, it is contemplated that any combination of these processes may occur in a particular embodiment of the present invention.

Another object of the present invention is to provide a solution for the diagnosis, prognosis, and treatment of anxiety that may be readily manufactured from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of the invention will be particularly pointed out in the claims, the invention itself and manners in which it may be made and used may be better understood after a review of the following description, taken in connection with the accompanying drawings, wherein like numeral annotations are provided throughout.

FIG. 1 depicts a flow schematic of a first exemplary embodiment of a system of the present invention.

FIG. 2 depicts a flow schematic of a second exemplary embodiment of a system of the present invention.

FIG. 3 depicts a schematic of an exemplary signal conversion device of the present invention.

### DETAILED DESCRIPTION OF THE INVENTION

Reference is made herein to the attached drawings. Like reference numerals are used throughout the drawings to depict like or similar elements of the invention. The figures are intended for representative purposes only and should not be considered limiting in any respect.

Reference is now made to the drawings, which depict one or more exemplary embodiments of the invention.

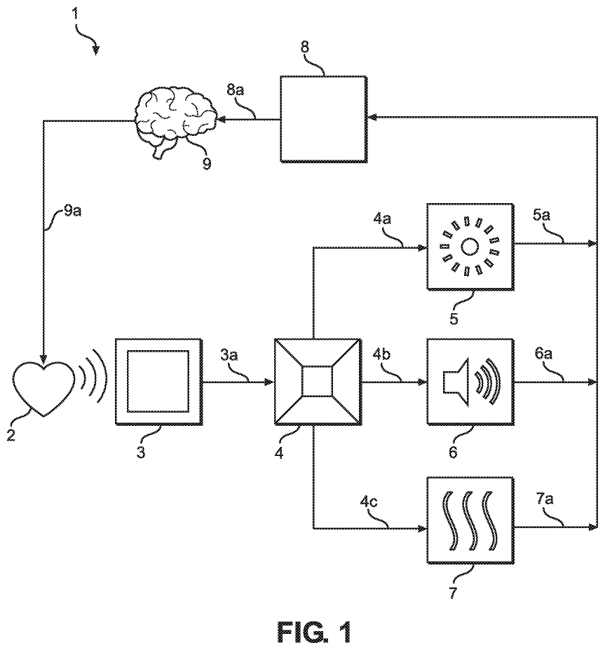

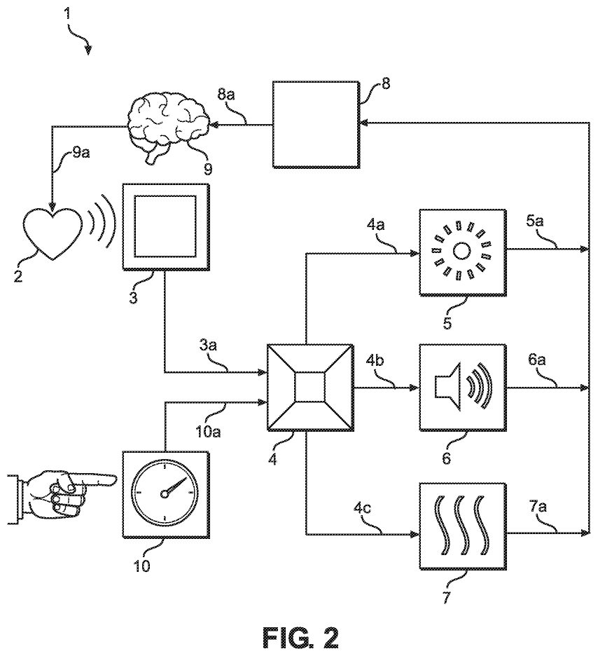

Referring now to FIGS. 1 and 2, there are depicted flow schematic of a first exemplary embodiment (FIG. 1) and a second exemplary embodiment (FIG. 2) of a system of the present invention. The system 1 includes a signal conversion device 3, which includes a sensor operably connected thereto and is configured to detect, measure, determine, or quantify a rate of a biological process, such as a rate of a heartbeat of a heart 2 of a first individual. In the shown embodiment, the signal conversion device 3 is operably connected to a computing device 4 by connection 3a. The computing device 4 processes a signal received from the signal conversion device 3 into one or more forms suitable for control of one or more biofeedback elements. In the shown embodiment, the system 1 includes a plurality of biofeedback elements, including a light element 5, a sound element 6, and a temperature element 7, however, in some embodiments, the system 1 includes one or more of the light element 5, the sound element 6, and/or the temperature element 7.

The computing device 4 is operably connected to the light element 5, the sound element 6, and the temperature element 7 by way of connections 4a, 4b, and 4c, respectively. The elements (5, 6, 7) produce outputs (5a, 6a, 7a) in the form of light (or the absence thereof) for the light element 5, sound (or the absence thereof) for the sound element 6, and/or a particular temperature for the temperature element 7. The biofeedback elements' outputs (5a, 6a, 7a) are integrated into an environment 8 containing or surrounding the first individual. The environment 8 may be referred to as an initial environment before a cycle of the system, and may be referred to as a resultant environment after the cycle of the system, as would be understood by the person having ordinary skill in the art. The sum total of sensory inputs from the environment 8 are transmitted from the environment 8 to one or more senses of the first individual by way of environmental connection 8a, and this sum total is experienced and/or processed by a nervous system 9 of the first individual. The nervous system 9 in turn controls the heartrate of the heart 2. In this manner, a biofeedback process is produced by the system, wherein the nervous system 9 of the first individual may have an ability to control, alter, or regulate the heartrate of the heart 2, or any mechanism involved in such regulation, according to a particular mode of action of the present invention.

In various embodiments, the signal conversion device 3 is configured to determine a property, such as a rate, of a biological process of an individual in a particular environment. In the shown embodiment, the biological process includes a heart pumping blood throughout a circulatory system of the individual, and the rate includes the rate of a pump cycle, as would be understood by a person having ordinary skill in the art. In some embodiments, the signal conversion device 3 may comprise one or more sensors, such as electrodes, configured to contact or be positioned near a skin of the individual to detect the heartbeat. In some embodiments, the sensors may be worn about a chest portion of the individual's body, but in alternate embodiments, the sensors may be worn about a wrist, an arm, a neck, or any other portion or area of the body which may be utilized to detect, measure, or determine the rate of the heartbeat. In this manner, it is contemplated that the present invention encompasses any and all structures and uses thereof for this purpose, including chest band type heartbeat monitors, as well as arm heartbeat monitors. In this manner, a comfort of the individual may be maximized or accommodated, and the accuracy of the detection, measurement, or determination may be ensured.

In various embodiments, the signal conversion device 3 includes elements of a computing system therein. These elements may include a logic executable by a circuitry and/or a processor, such as a microprocessor, of the signal conversion device 3. The executable logic may be at least partially stored on a non-transitory computer-readable medium, such as a stationary memory or storage volume. In such embodiments, the signal conversion device 3 may include a non-transitory computer-readable storage volume for the storage and retrieval of data obtained by the signal conversion device 3 that pertains to the biological process being monitored, detected, measured, or otherwise determined. In various embodiments, the signal conversion device 3 includes a wired and/or a wireless signal transceiver or transmitter.

In some embodiments, the signal conversion device 3 is configured for use with a Bluetooth® wireless radio signal. In some embodiments, the signal conversion device 3 is configured to transmit a wireless radio signal having a range of up to and including fifty (50) feet. In this manner, the biological process may be remotely monitored by a third party, such as a clinician performing tests or psychiatric evaluations for the individual wearing the signal conversion device, without interfering with or disturbing the individual(s) under observation.

In various embodiments, the connection 3a between the signal conversion device 3 and the computing device 4 includes a wired connection. In this manner, the computing device 4 may be worn along with the signal conversion device 3, which may be preferred in particular scenarios or situations. However, in some embodiments, the connection 3a between the signal conversion device 3 and the computing device 4 includes a wireless connection, which may provide several advantages. For example, a wireless connection 3a would enable the signal conversion device 3 to be constructed of a relatively low profile shape or form factor, enhancing comfort experienced by the individual when the signal conversion device 3 is worn. Further, the wireless connection 3a may enable greater variety with respect to the exact type of computing device 4 utilized in a particular scenario. For example, in various embodiments, one or more devices such as a smartphone, a smart watch, a personal computer, a laptop computer, a tablet computer, and the like, may be utilized for monitoring a signal received from the signal conversion device 3 via the connection 3a.

Generally, the purpose of the signal conversion device 3 is to convert a biological signal into an electronic signal that can be further processed by the computing device 4. The biological signal may be a signal received from an electrical activity of the heart 2, and in this case, the signal conversion device 3 converts the biological signal into the electronic signal which is communicated by way of the connection 3a. The electronic signal produced may be analog or digital, according to need.

In some embodiments, the logic of the signal conversion device 3 converts the biological signal to the electronic signal according to a conversion factor. Such a conversion factor may be fixed or variable, and in general is configured to enable the computing device 4 to monitor the electronic signal and produce a desired output, according to a particular scenario, measurement, or determination. In the exemplary embodiment of a heartbeat being monitored, the conversion factor may be used to define an upper limit and a lower limit for the heartbeat before a particular output is initiated, stopped, or altered. For example, the upper limit may correspond to a high beat per minute (bpm) measurement, which may map to a high note in a musical output, and the lower limit may correspond to a low bpm, which may map to a low note in the musical output. In this example, the intervening heart rates (in bpm) may be divided into sub-ranges which correspond to notes in a scale between the low note and the high note.

In some embodiments, the conversion factor is variable according to an average or a moving average for a particular individual monitored by the signal conversion device 3. Accordingly, it is envisioned that certain ratios of an average for a particular individual may be utilized to produce the conversion factor. In this manner, a person having a higher than average resting rate for a particular biological process may produce the same or a similar result as a person having a lower than average resting rate for a particular biological process. For example, if 90 bpm is an average of a first person's heart rate, and if 60 bpm is an average of a second person's heart rate, then the output of each of these persons would be the same (i.e., the same behavior of one or more of the biofeedback elements 5a, 6a, 7a) if the heart rate of the first person is 90 bpm and if the heart rate of the second person is 60 bpm. Similarly, an upper value for the first person may differ in bpm from an upper value for the second person without differing in effective output of one or more of the biofeedback elements 5a, 6a, 7a.

In various embodiments, the computing device 4 may be designed, selected, and/or configured to process a signal received from connection 3a and convert the signal into biofeedback element signals to be sent along connections 4a, 4b, and/or 4c. For example, in various embodiments, one or more devices such as a smartphone, a smart watch, a personal computer, a laptop computer, a tablet computer, and the like, may be utilized as the computing device 4 for monitoring and processing the signal received from the signal conversion device 3 via the connection 3a. In this manner, the signal conversion device 3 may be used, combined, or integrated with any particular computing device according to need.

In various embodiments, the computing device 4 includes elements of a computing system therein. These elements may include a logic executable by a circuitry and/or a processor, such as a microprocessor, of the computing device 4. The executable logic may be at least partially stored on a non-transitory computer-readable medium, such as a stationary memory or storage volume. In such embodiments, the computing device 4 may include a non-transitory computer-readable storage volume for the storage and retrieval of data obtained by the computing device 4 that pertains to the biological process being monitored, detected, measured, or otherwise determined. In various embodiments, the computing device 3 includes a wired and/or a wireless transceiver or transmitter.

In the shown embodiment, the computing device 4 is operably coupled to the light element 5, the sound element 6, and the temperature element 7, via connections 4a, 4b, and 4c, respectively, and in this manner the system 1 includes the light element 5, the sound element 6, and the temperature element 7. However, in alternate embodiments, the system 1 includes the light element 5 without the sound element 6 or the temperature element 7. In alternate embodiments, the system 1 includes the sound element 6 without the light element 5 or the temperature element 7. In alternate embodiments, the system 1 includes the temperature element 7 without the light element 5 or the sound element 6. In some embodiments, the system 1 includes the light element 5 and the sound element 6 without the temperature element 7. In some embodiments, the system 1 includes the light element 5 and the temperature element 7 without the sound element 6. In some embodiments the system 1 includes the light element 5 and the temperature element 7 without the sound element 6. In this manner, the exact design of the system 1 may be customizable at least through the selection of particular biofeedback elements to include.

In the shown embodiment, the signal conversion device 3 is a separate or distinct device from the computing device 4. In this manner, the signal conversion device 3 may be housed in a housing that is separate or distinct from a housing that houses the computing device 4. However, in alternate embodiments, the signal conversion device 3 may be housed within a common housing that also contains at least the computing device 4. In like manner, the biofeedback elements may also be commonly housed or may be separate or distinct from the other components.

Generally, the light element 5 may include one or more lights. The one or more lights may be any suitable light, however, it may be desirable to include one or more lights that can be altered in some way by a signal from connection 4a. In some embodiments, one or more light emitting diodes (LEDs) may be utilized for the light element 5. In some embodiments, an ultraviolet (UV) lamp may be utilized for the light element 5. In some embodiments, a heat given off by a light of the light element 5 may provide a temperature of the temperature element 7, and in this manner, both heat and light may be combined as a dual biofeedback element. In some embodiments, a wavelength or frequency of light emitted from one or more lights of the one or more lights may be altered by the signal from connection 4a. In some embodiments, an intensity of light emitted from one or more lights of the one or more lights may be altered by the signal from connection 4a.

Generally, the sound element may include one or more audio speakers. The one or more audio speakers may be configured to receive an analog or a digital signal from the computing device 4 via the connection 4b. In this manner, the connection 4b may be wired or wireless, according to a particular need or design consideration. The content of the signal communicated along connection 4b may include data pertaining to a song, a tone, or a tone of the song. In some embodiments, a speed of a song being played through the one or more audio speakers may be adjusted according to a rate of the biological process, e.g., the heart rate of the heart 2. In this manner, the individual emitting the heart rate and hearing a sound of the one or more audio speakers may be able to control his or her heart rate.

Generally, the temperature element 7 may include one or more heating elements or cooling elements, as needed, to adjust a temperature of the environment 8. In some embodiments, this may be a part of a hot or cold therapeutic regimen, and the temperature emanating from the temperature element 7 may be sensed, felt, or applied directly to a portion of the body of the individual. In this manner, the therapeutic regimen may be altered through one or more biofeedback mechanisms.

Signals 5a, 6a, and 7a from the biofeedback elements (5, 6, 7) are integrated into the environment 8 containing the individual. These signals become a portion, such as a small portion, an average portion, or a large portion, of the environment experienced by the individual. In some embodiments, the individual may be wearing sound-canceling ear buds or headphones for the sound element 6, and in such scenarios, an ambient sound of the environment 8 may constitute a small fraction of the sound of the environment 8. Likewise, in some embodiments, the environment may be largely dark except for a light emanating from the light element 5, and in such scenarios, an ambient light of the environment 8 may constitute a small fraction of the light of the environment 8. In this manner, for certain scenarios or circumstances, the biofeedback elements may comprise a large majority of the environment 8, or alternately, may comprise a small minority of the environment 8, or anywhere therebetween.

Elements of the environment 8 which are detectable by senses are sent to the nervous system 9 of the individual by connection 8a, which may be accomplished through sensory input of one or more organs of the individual, such as an eye, an ear, and/or a portion of skin. Because anxiety may be exacerbated by substantial or uncontrollable environmental stimuli, the environment 8 may be effectively controlled by the system 1 so as to reduce a likelihood of an undesired symptom of anxiety. After the environment 8 is sensed by the nervous system 9 of the individual by connection 8a, the nervous system 9 may attempt or may successfully control, alter, or augment an activity of the heart 2 of the individual, including but not limited to a heart rate of the heart 2 of the individual. The updated, changed, or altered heart rate is then sensed by the sensor of the signal conversion device 3, and a process or use of the system 1 may be repeated.

Referring now to FIG. 2, there is depicted a flow schematic of a second exemplary embodiment of a system of the present invention. The shown embodiment is similar to the embodiment of FIG. 1, but in the shown embodiment, the system 1 is configured to accept an input from the first individual having the heart 2, as well as an input from a second individual, via connection 10a. In the shown embodiment, the connection 10a accepts an input that may be consciously or manually controlled by the second individual, such as by a switch or a dial (i.e., control 10). The control 10 may be used to alter or override a signal of the connection 3a to dampen, augment, cancel out, or silence one or more components of the environment 8 by altering or overriding one or more outputs of the biofeedback elements (5, 6, 7). For example, if a first individual having the heart 2 begins experiencing symptoms of anxiety or panic, a caretaker may utilize the control 10 to alter the biofeedback elements (5, 6, 7) by providing the signal via connection 10a to the computing device 4. In this manner, the first individual may avoid having a panic attack or other psychiatric event. In some embodiments, the first individual may control the control 10 themselves. This may be particularly useful if the first individual is unable to control their anxiety in a particular setting and immediate positive or therapeutic environment is needed.

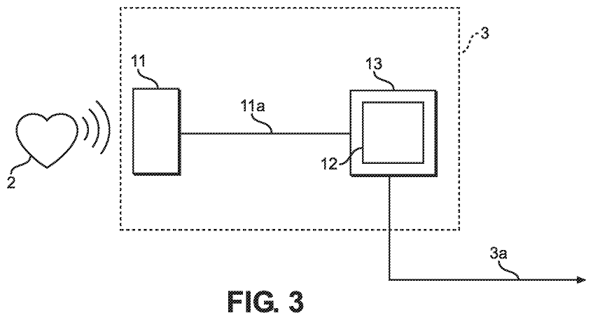

Referring now to FIG. 3, there is depicted a schematic of an exemplary signal conversion device of the present invention. Generally, the purpose of the signal conversion device 3 is to convert a biological signal, e.g., a heartbeat of the heart 2, into an electronic signal, e.g., a signal to be communicated via the connection 3a, that can be further processed by the computing device to produce one or more signals from one or more biofeedback elements. In the shown embodiment, the signal conversion device 3 includes a sensor 11, a processing unit 12, and a circuitry substrate 13. In the shown embodiment, the sensor 11 is configured to detect or measure a heart beat or heart rate. The sensor 11 is connected to the circuit substrate 13 by way of connection 11a. In an exemplary embodiment, the sensor 1 is a Polar® T-34 heart monitor, the processing unit 12 is an Arduino® unit, such as a Teensy 3.2 unit, and the circuitry substrate 13 is a printed circuit board (PCB).

In various embodiments, the processing unit 12 may be programmed according to a logic on a non-transitory computer readable medium thereof, and this logic may control one or more aspects of the conversion of the biological signal to the electronic signal. In various embodiments, the circuitry substrate 13 may include a wired connection for connection 3a and/or 11a. In other embodiments, the circuitry substrate 13 may include a wireless connection for connection 3a and/or 11a.

In the shown embodiment, the sensor 11 is wirelessly connected to the circuitry substrate 12 by connection 11a, wherein connection 11a is wireless. In the shown embodiment, the sensor includes a wireless transceiver or transmitter therein, which may include but is not necessarily limited to a Bluetooth® transceiver or transmitter, for establishment of connection 11a. In like manner, the circuitry substrate 13 may include a wireless transceiver or receiver connected thereto, and the connection 11a may be established thereby. In this manner, the connection 11a may be made according to any suitable means. In some embodiments a range of the connection 11a may exceed a range of an ordinary Bluetooth® connection, and in this manner, the sensor 11 may be worn by the first individual and observed by the second individual or a third individual from a remote distance, including through one or more walls or windows. In some embodiments, the range of the connection 11a may include any range up to and including fifty feet (50 ft.), or more, according to a particular embodiment.

In the shown embodiment, the processing unit 12 is operably connected to the circuitry substrate 13, which operably connects the processing unit to the sensor 11. In like manner, the processing unit 12 is operably connected to the connection 3a by way of the circuitry substrate 13. In this manner, the circuitry substrate connects the processing unit 12, the wired or wireless transceiver, receiver, and/or transmitter of the circuitry substrate 13 to the connection 11a and the connection 3a, and as a result, the sensor 11 and the computing device, as described elsewhere herein.

Generally, the system of the present invention may comprise the device (i.e., the signal conversion device), and may be used in a method of therapy, such as music therapy. As described elsewhere herein, music therapy is advantageous for a number of reasons, but it may be desired to alter a particular approach for music therapy with one or more biofeedback elements, as described elsewhere herein. In such scenarios the present invention is particularly useful or advantageous, at least because the music of the music therapy may effectively be controlled by the heartbeat of the heart of the individual, which in turn may be controlled at least in part through a conscious effort or approach. This method of use may be particularly advantageous for diagnosis, prognosis, and/or treatment of individuals with anxiety or related conditions.

The foregoing descriptions of specific embodiments of the present invention have been presented for purposes of illustration and description. They are not intended to be exhaustive or to limit the present invention to the precise forms disclosed, and modifications and variations are possible in view of the above teaching. The exemplary embodiment was chosen and described to best explain the principles of the present invention and its practical application, to thereby enable others skilled in the art to best utilize the present invention and its embodiments with modifications as suited to the use contemplated.

It is therefore submitted that the present invention has been shown and described in the most practical and exemplary embodiments. It should be recognized that departures may be made which fall within the scope of the invention. With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention.

I claim:

1) A signal conversion device for diagnosis, prognosis, or treatment of a disease or disorder of a first individual, comprising:
a sensor configured to sense a first biological process of the first individual;
a processor configured to execute a logic stored on a non-transitory computer-readable medium;
wherein the logic is configured to:
determine a rate of the first biological process of the first individual in an initial environment;
convert the rate into a frequency according to a conversion factor;
transmit a signal from the signal conversion device to a computing device via a signal transmitter operably connected to the signal conversion device;
wherein the computing device is configured to process the signal received from the signal conversion device and regulate one or more properties of one or more biofeedback elements to alter an initial environment containing the first individual to produce a resultant environment containing the first individual;
wherein a difference between the resultant environment and the initial environment is perceptible to at least the first individual.

2) The signal conversion device of claim 1, wherein the resultant environment influences the first biological process of the first individual.

3) The signal conversion device of claim 1, wherein the signal conversion device is further configured to regulate one or more properties of the signal according to a manual input of the first individual or a manual input of a second individual.

4) The signal conversion device of claim 3, wherein the manual input is configured to operate independently of the first biological process.

5) The signal conversion device of claim 1, further comprising a second device configured to regulate one or more properties of the resultant environment according to an input of the first individual or an input of a second individual.

6) The signal conversion device of claim 5, wherein one or more of the input of the first individual and the input of the second individual includes a manual input.

7) The signal conversion device of claim 6, wherein the manual input is configured to operate independently of the first biological process.

8) A system for treatment of a disease or disorder of a first individual characterized at least in part by anxiety, comprising the signal conversion device of claim 1.

9) The system of claim 8, wherein the resultant environment influences the first biological process of the first individual.

10) A method for treatment of a disease or disorder of a first individual characterized at least in part by anxiety, comprising:
determining a heart rate of the first individual in an initial environment;
converting the heart rate into a frequency according to a conversion factor;
processing the frequency to regulate one or more properties of one or more biofeedback elements to alter the initial environment containing the first individual to produce a resultant environment containing the first individual;
wherein the one or more biofeedback elements is selected from a group consisting of: a light element, a sound element, a temperature element, and a combination thereof.

11) The method of claim 10, wherein the resultant environment influences the first biological process of the first individual.

12) The method of claim 10, further comprising regulating one or more properties of the one or more biofeedback elements according to a manual input of the first individual or a manual input of a second individual.

13) The method of claim 12, wherein the manual input is configured to operate independently of the heart rate of the first individual.

14) A method of performing music therapy for treatment of anxiety, comprising:
determining a heart rate of a first individual in an initial environment;
converting the heart rate into a frequency according to a conversion factor;
processing the frequency to regulate one or more properties of one or more biofeedback elements to alter the initial environment containing the first individual to produce a resultant environment containing the first individual;
wherein the one or more biofeedback elements includes a sound element;
wherein the one or more properties of the sound element is selected from a group consisting of: a pitch, a duration, a loudness, a timbre, a sonic texture, a spatial location, and a combination thereof;
wherein the resultant environment influences the first biological process of the first individual.
